(19) United States
(12) Patent Application Publication
(10) Pub. No.: US 2009/0226996 A1
(43) Pub. Date: Sep. 10, 2009

(54) Oxygen scavenger coating composition

(76) Inventors: Dexter A. Chisholm, Unionville, PA (US); John Henry Green, Oxford, PA (US); Noel M. Hasty, Wilmington, DE (US); Alexander Vincent Tobias, Hockessin, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 12/074,432

(22) Filed: Mar. 4, 2008

(51) Int. Cl. C12N 9/00

(52) U.S. Cl. 435/190; 435/183

(57) ABSTRACT

A coating composition having an multicopper oxidase enzyme and an oxidizable substrate in combination with an organic binder polymer which may be dispersed or dissolved in a non-aqueous liquid vehicle.

### FIELD OF INVENTION

The invention is directed to an organic composition comprising iron suitable for use in the preparation of oxygen scavenging films, sheets, and layers. More specifically, the invention provides a non-aqueous liquid vehicle based coating composition comprising an enzyme, an oxidizable substrate, iron, and an organic polymer binder.

### BACKGROUND

Oxidative degradation of packaged goods has long been recognized as a problem affecting both appearance and useful life. Such goods include, for example, food, beverages, cosmetics, personal care products, electronic components/devices and pharmaceuticals.

Enzyme-substrate systems have long been known in the art as effective oxygen scavenging systems. Such systems include a combination of glucose oxidase and glucose; and the use of glucose oxidase, catylase, and glucose impregnated into a sheet used to wrap a packaged good. Other systems include the use of glucose oxidase, catylase, glucose and a water-soluble polymeric binder; and enzyme and substrate incorporated as a thin film between multiple layers. Ascorbate oxidase has been disclosed as an additive to ascorbate-containing foods and juices. Ascorbate oxidase in the form of an immobilized enzyme covalently bound to the inner lining of food packages has been disclosed. Laccase is an oxidase with a wide substrate range and is used as a deoxygenating food additive, where naturally occurring reducing substrates are used by laccase to convert oxygen to water.

U.S. published patent application 2005/0205840 to Farneth et al. discloses a process to remove oxygen from a sealed container wherein an O₂ scavenging system is provided comprising an enzyme and a reducing substrate. The system as described in Farneth is an easily applied water-based formulation. However, the system of Farneth actively scavenges oxygen during storage and while being applied to the container, squandering scavenging capacity and activity.

Yeh et al., J. Polym. Engg. (2007), 27 (4), 245-265, discloses use of compositions of metallic iron and ascorbic acid for scavenging of oxygen.

An oxygen scavenging composition desirably provides rapid reduction of oxygen concentration after package sealing combined with sufficient capacity to maintain reduced oxygen concentration over weeks or months of storage. There is a need for an non-aqueous liquid vehicle based composition that allows the scavenger system to remain inert during formulation, transport, storage, and application in making films, sheets or layers for oxygen scavenging articles.

### SUMMARY OF THE INVENTION

The present invention is directed to a composition comprising a non-aqueous liquid vehicle, a multicopper oxidase enzyme, an oxidizable substrate, metallic iron, and an organic binder polymer wherein the polymer is dissolved or dispersed in the liquid vehicle, and wherein the enzyme, substrate and metallic iron are in particulate form and dispersed in the liquid vehicle.

### DETAILED DESCRIPTION OF THE INVENTION

The oxygen scavenging composition employed herein—combining multicopper oxidase enzyme, oxidizable substrate, and iron—exhibits a rate of oxygen scavenging that is well in excess of that predicted from the rule of mixtures applied to the combination of enzyme/substrate and iron/substrate compositions that are known in the art.

For the purposes of the present invention, the term “substrate” is employed according to usage in the biochemical art to refer to a redox reactive reagent and carries with it no reference to any particular form, shape, size, or any morphological consideration. An oxidizable substrate is a reagent which will undergo oxidation. It will react with oxygen in a reaction which is catalyzed by an enzyme and is synonymous with the term “reductant”.

The present invention provides a composition suitable for use in preparing an oxygen scavenging coating composition comprising an organic binder polymer, a multicopper oxidase enzyme, an oxidizable substrate, and iron, wherein the multicopper oxidase enzyme, the oxidizable substrate, and iron are sufficiently intermixed to allow oxidation of the substrate in the presence of molecular oxygen. The composition utilizes a non-aqueous liquid vehicle.

In an embodiment the multicopper oxidase enzyme, the oxidizable substrate, and iron are in particulate form wherein they are combined to form a mixture of particles of oxidizable substrate, particles of multicopper oxidase enzyme, and particles of iron. In another embodiment, the oxidizable substrate and iron are an intermixed particulate form wherein multicopper oxidase enzyme is disposed upon the surfaces of the substrate particles. In another embodiment, the multicopper oxidase enzyme, the oxidizable substrate, and iron are in particulate form wherein the enzyme is dispersed throughout the body of the substrate particles. The multicopper oxidase is selected from laccase or ascorbate oxidase. The average equivalent spherical diameter of the particles of the oxidizable substrate, and iron as determined by light scattering techniques ranges from 1 to 100 micrometers, and preferably, 1 to 20 micrometers.

Multicopper oxidase enzymes are suitable for use in the present invention. Examples include laccase and ascorbate oxidase.

Laccase (E.C. 1.10.3.2, Systematic Name: Benzenediol:oxygen oxidoreductase) and ascorbate oxidase (E.C. 1.10.3.3 Systematic Name: L-ascorbate:oxygen oxidoreductase) are two classes of multicopper oxido-reductases which perform—in combination with a suitable substrate—a four-electron reduction of molecular O₂ to form H₂O.

Laccases occur in plants, fungi, yeasts and bacteria. Best known laccase producers are fungi. Fungal laccases suitable for the purposes of the present invention herein include (but are not limited to) those isolated from Ascomycetes and Basidiomycetes. More specifically, illustrative sources of fungal laccases include those from: Aspergillus, Neurospora, Podospora, Botrytis, Collybia, Fomes, Lentinus, Pleurotus, Trametes, Rhizoctonia, Coprinus, Psaturella, Myceliophthora, Schytalidium, Polyporus, Phlebia, Coriolus, Hydrophoropsis, Agaricus, Cascellum, Crucibulum, Myrothecium, Stachybotrys, Sporormiella, Trametes versicolor, T. villosa, Myceliophthora thermophilia, Stachybotrys chartarum, Coriolus hirsutus and C. versicolor. Commercially available laccases are available from sources such as Wacker Chemie (München, Germany; T. versicolor), Novozymes (Franklinton, NC; M. thermophilia), Genencor (Palo Alto, Calif.; S. chartarum), Sigma-Aldrich (St. Louis, Mo.; C. versicolor) and SynectiQ (Dover, N.J.; C. hirsutus).

The source of laccase is not limiting to the invention. Thus, although fungal laccases are preferred, laccases can also be obtained from transgenic yeasts (e.g., Pichia, Saccharomyces and Kluyveromyces), transgenic fungi (e.g., Aspergillus, Trichoderma or Chrysosporium) and transgenic plants that serve as production hosts to express laccase genes cloned from other organisms (e.g., of fungal origin). Additionally, laccase may be produced from a variety of bacteria (e.g., Escherichia, Bacillus and Streptomyces).

Additionally non-native laccases may, also, be used in the invention. These modified laccases can be obtained by traditional mutagenesis (e.g., chemical, UV) or directed evolution methods (e.g., in vitro mutagenesis and selection, site-directed mutagenesis, error prone PCR, “gene shuffling”), wherein the techniques are designed to alter the amino acid sequence of the protein with the objective of improving the characteristics of the laccase. Examples of improvements would include altering substrate specificity or increasing the stability of the native enzyme.

For a general review of ascorbate oxidases, see for example: Dawson, C. R., K. G. Strothkamp and K. G. Krul. Ann NY Acad Sci. 258:209-220 (1975).

Ascorbate oxidases are known to originate from plants. Ascorbate oxidases suitable for the purposes of the present invention include (but are not limited to) those isolated from tobacco, soybean, cucumber, squash plants, etc. More preferred, however, are those thermally stable ascorbate oxidases that are isolated from fungi, and in particular, from species of the genus Acremonium (e.g., see U.S. Pat. No, 5,180,672).

For the purposes of the present invention, suitable reducing substrates—or, synonymously, oxidizable substrates or reductants—are compounds that are capable of donating electrons to the type 1 copper site of a multicopper oxidase, such as laccase or ascorbate oxidase. Laccase is well known to be able to accept electrons from a wide range of phenolic molecules such as flavonoids and quinones, as well as some small non-phenolic molecules. Substrates include ascorbic acid and salts thereof, such as calcium ascorbate or sodium ascorbate, isoascorbic acid and salts thereof, and combinations thereof.

In an embodiment, substrate particles having a multicopper oxidase enzyme disposed upon the surfaces thereof are first prepared, and then combined with iron particles to form the composition hereof. In another embodiment, substrate particles having the multicopper oxidase enzyme dispersed throughout the body thereof are first prepared, then combined with iron particles to form the composition hereof.

The average equivalent spherical diameter of the particles of the oxidizable substrate as determined by light scattering techniques ranges from 1 to 100 micrometers, and preferably, 1 to 20 micrometers. The iron suitable for use in the present invention is metallic iron in particulate form, with average particle size in the range of 10 nm to 100 μm, preferably 100 nm to 50 μm. Suitable iron particles are widely available commercially from, among others, BASF, North American Höganäs, Toda Kogyo, and Alfa Aesar.

The relative amounts of the ingredients of the composition in any specific embodiment hereof will be dictated by the particular requisites of the particular use for which it is intended. The relative concentration of the multicopper oxidase enzyme and the oxidizable substrate range from one part by weight of the enzyme combined with 20 to 1000, preferably 50 to 500, parts by weight of the oxidizable substrate.

There is no particular limit on the relative concentrations of iron and the enzyme/substrate in the composition hereof. The weight ratio of iron to enzyme substrate may range from 5:95 to 95:5. It is found in the practice of the invention that compositions having 15-25% by weight of iron particles exhibit the greatest enhancement in the rate of oxygen removal in a closed package.

Although the particular morphology of the mixture hereof will influence performance details in particular situations, no one particular way of preparing the composition hereof is preferred over another. The critical factor in preparing the compositions hereof is to prepare a morphology that, upon introduction of moisture thereto enables the oxygen scavenging chemistry to proceed. While the invention hereof is in no way limited to any particular chemical mechanism, it is speculated by the inventors hereof that several chemical reactions may be occurring simultaneously. The may include the enzyme catalyzed reaction of the substrate with oxygen in aqueous solution, the aqueous salt activated reaction of iron with oxygen, also in aqueous solution, and the iron activated reaction of the substrate with oxygen, also in aqueous solution.

Any method or combination of methods, for preparing a mixture of the multicopper oxidase enzyme, the oxidizable substrate, and the iron may be employed. In the process for preparing the composition hereof, it is preferred to combine the enzyme and substrate mixture before adding in the iron therewith. However, the process for preparation of the composition hereof is not thereto limited. Combination of enzyme and substrate is accomplished advantageously in aqueous solution, but may also be performed in non-aqueous dispersions thereof, such as in alcohol. However the enzyme/substrate mixture is effected, it is preferred to disperse the resulting composition in particulate form with the iron particulates in a non-aqueous vehicle such as but not limited to ethanol.

In one embodiment, the enzyme and substrate are combined in aqueous solution, followed by drying and milling to produce fine particles, in the range of 1-100 micrometers in size. Spray drying is one method of producing small homogeneous particles which minimize the amount of grinding needed to achieve a desired particle size. After thus combining the enzyme and substrate, the iron may be combined therewith.

In another embodiment, a mixture is formed by subjecting particles of the substrate to contact with an aqueous solution of the enzyme. Many methods for performing the requisite contacting operation are known including drum coating, pan coating, fluidized bed drying, fluidized bed mixing, v-cone blending, and injector treatment methods. The substrate particles may be first ground as necessary into the 1-100 micrometer size range, prior to contacting with the enzyme solution. In the alternative, the enzyme coated particles may be subject to comminution after treatment. After thus combining the enzyme and substrate, the iron may be combined therewith.

The coating or ink compositions hereof further comprise a non-aqueous liquid vehicle and an organic binder polymer with the proviso that the multicopper oxidase enzyme, the oxidizable substrate, and the iron are sufficiently intermixed to allow oxidation of the substrate and the iron in the presence of molecular oxygen upon exposure to moisture

In another embodiment the substrate particles are subject to milling in which the vehicle is the grinding medium. Enzyme in powder form or concentrated aqueous solution can be added during milling to produce dispersion coated substrate particles. The particles so-produced are then dispersed with iron particles to form the composition hereof.

Alternatively, it is possible to prepare the composition hereof by combining the components by direct addition of each to the vehicle while mixing. The loading volumes of the reactive components (the enzyme, substrate, and iron) with respect to the binder polymer and other components need to be high enough so that the active components are sufficiently intermixed in the final coating that they are able to chemically react. In general, the ingredients may be combined in any order.

In an embodiment, the composition hereof is prepared and then combined with the vehicle. The dispersion of the composition hereof in the vehicle can be accomplished by using a high-speed disperser, sand mill, bead mill, or media mill. The concentration of dispersed particles in the vehicle ranges from 0.1% by weight to about 33% by weight of the total composition.

In an embodiment, the organic binder polymer is water-insoluble. The invention encompasses embodiments wherein the polymer is not soluble in the non-aqueous liquid vehicle composition. In those embodiments, the coating or ink composition comprises a dispersion of the polymer particles with the composition hereof in particulate form in the non-aqueous liquid vehicle. In another embodiment, the composition comprises a dispersion of the composition hereof in a solution of the polymer and non-aqueous liquid vehicle.

The discussion following is directed to embodiments wherein the polymer is soluble in the non-aqueous liquid vehicle.

The choice of vehicle will depend largely upon the requirements of a specific application. There is no limitation on the choice of vehicle so long as it is selected to be compatible with the polymeric binder. Suitable vehicles include but are not limited to ethyl acetate, ethanol, toluene, tetrahydrofuran (THF), methyl ethyl ketone, isopropyl alcohol, dibasic esters, 2-ethyl-hexyl acetate, normal propyl acetate, n-butyl acetate, isopropyl acetate, dimethyl formamide, N-methyl pyrolodone, acetone, cyclohexane, ethylene glycol diacetate, and mixtures thereof.

In one embodiment, the coating composition is employed for food packaging, so compatibility of the vehicle and the polymer with food is important. In typical use, the ink or coating composition is coated onto a surface, and the vehicle is evaporated leaving behind a solid coating or film on the surface. Although the vehicle is removed prior to use of the oxygen scavenging film, food contact by trace amounts of vehicle cannot be excluded as a possibility. The package can be designed so that the activated oxygen scavenging coated surface is in diffusive oxygen contact with the food item, but is prevented from actual physical contact by virtue of compartmentalization, or a physical barrier. A physical barrier may be an oxygen and moisture permeable film laminated, extrusion coated or solvent coated onto the activated oxygen scavenging film.

In another embodiment, the coating composition is applied to a surface by printing. Compatibility with or inertness to the printing surface is necessary.

Polymers useful in the invention may or may not be soluble in the vehicle. Polymers insoluble in the vehicle are less preferred. Water insoluble polymers impart strength and structural integrity to the resultant scavenging film, especially after long exposure to high humidity. Water insoluble polymers may exhibit significant water uptake or water permeability. In contrast, water soluble polymers become slimy and lose film strength when in a moist environment; slimy polymers may be distressing to the consumer when the composition of the invention is employed to prepare, e.g., consumer-directed packaging as in packaged meats.

Suitable polymers are either soluble or dispersible in the selected vehicle and are capable of forming a film or layer, upon deposition and evaporation of the vehicle. The particular selection of polymer will depend upon the suitability for a particular use. Polymers soluble in a vehicle are preferred. In an embodiment of the invention intended for use in food packaging, the polymer should be safe to use with food. If rapid oxygen scavenging is desired, then the polymer should be permeable to oxygen and water vapor. Examples of suitable binder polymers include but are not limited to ethylene vinyl acetate copolymers or terpolymers, such as Elvax® or Elavaloy® available from DuPont; cellulosic polymers, such as cellulose acetate, cellulose acetate propionate, or cellulose acetate butyrate; acrylic polymers, such as poly(butylmethacrylate) or poly(butyl methacrylate-co-methyl methacrylate); polyurethanes prepared by reacting excess aliphatic diisocyanate with polyether or polyester polyol, diamine and terminating agent; cosolvent polyamides; and polyesters, such as polyethylene sebacate or poly(butylene adipate); or mixtures thereof.

In one embodiment, one part by weight of the multicopper oxidase enzyme and the oxidizable substrate is then combined with 0.05 to 20 parts by weight of the polymer in the vehicle. In one embodiment, the polymer is insoluble in the non-aqueous liquid vehicle so that the coating or ink composition hereof comprises a dispersion of both the polymer and the multicopper oxidase enzyme, the oxidizable substrate, and iron in the non-aqueous liquid vehicle. In another embodiment, the polymer is soluble in the vehicle, and the coating or ink composition hereof comprises a dispersion of the multicopper oxidase enzyme, the oxidizable substrate, and iron in a solution of the polymer and non-aqueous liquid vehicle. In an alternative embodiment, the enzyme resides on or is disposed upon the surface of the oxidizable substrate. In another embodiment, the enzyme is dispersed within the oxidizable substrate.

The discussion following is directed to embodiments wherein the polymer is soluble in the non-aqueous liquid vehicle. However, it shall be understood that the discussion applies to embodiments in which the polymer is dispersed as particles in the vehicle.

The choice of vehicle will depend largely upon the requirements of a specific application.

One part by weight of the enzyme is combined with 20 to 1000, preferably 50 to 500, parts by weight of the oxidizable substrate. In a typical application, one part by weight of the oxidizable substrate is combined in the vehicle with 0.05 to 20 parts by weight of the polymer. Iron is then added in a ratio to the oxidizable substrate ranging from 5:95 to 95:5 by weight, preferably the iron concentration is 15-25% by weight of the total iron, enzyme, substrate composition.

As a general rule, the least amount of polymer should be employed consistent with viscosity considerations and the degree of binding necessary. For a given amount of substrate, there are advantages to higher substrate to polymer ratios. Among these are that a given volume of ink will have higher scavenging capacity, the materials cost for a given scavenger capacity is lower, polymers with limited solubility in the vehicle will still dissolve, and the polymers employed may be less permeable to oxygen or water while still permitting a useful coating.

Other additives may include hygroscopic agents, such as fructose, silica gel, or polyvinyl alcohol; plasticizers soluble in the polymer; dispersing agents, such as Tween® 80, Triton® X-100 and Pluronic®; pigments, and such others that are commonly employed in the art for modifying the properties of polymers and inks. A dispersing agent helps to form a suspending medium promoting uniform and maximum separation of the fine solid particles.

In a typical application, a surface is contacted with a composition comprising a mixture of the vehicle, the polymer, and the composition hereof dispersed there-within. The vehicle is then evaporated thereby disposing upon the surface the composition in the form of a film, sheet, or layer.

In a typical use, the composition is applied to a surface, such as the inner surface of a food package, and the vehicle evaporated leaving behind a coating comprising a polymeric matrix binding the particles of the composition hereof. In the presence of moisture or water vapor the oxygen scavenging reaction is activated. The ink or coating composition of the invention can be applied to a surface according to methods well-known in the art. Examples of suitable surfaces include: wood pulp filter paper, glass fiber filter paper, paperboard, fabric, nonwoven fabrics, polymer films, metal foils, and label stock. Examples of suitable methods of application of the composition include solution-casting, spraying, blotting, knife over roll coating, curtain coating, dip coating, metering rod coating, reverse roll coating, painting with an applicator, and printing techniques including gravure, screen, ink jet, and flexographic.

In other applications, dispersions of the composition of the invention make excellent printing inks. The inks can be formulated to a suitable viscosity for screen printing, gravure printing, or flexographic printing. Additives, such as fillers or pigments may be incorporated without disturbing functionality.

The vehicle employed in forming the ink or coating composition can be selected to allow the compositions to be formulated well in advance of use. In some embodiments, the dispersions are stable over periods of months retaining the scavenging capacity. In the unactivated state of the unhydrated dispersed particles, the ink composition is unreactive with atmospheric oxygen which allows printing on unmodified equipment. Furthermore, fast-drying non-aqueous vehicles enable the use of high speed printing methods.

To prepare an ink formulation, particles of the composition hereof are dispersed in a vehicle using a media mill, sand mill, or high speed disperser forming a dispersion. The dispersion should contain 40%-70% by weight of scavenging particles, preferably 55% to 60%; an amount of dispersant may be added equal to ½ to 1/10 the weight of scavenging particles, preferably ¼ to ⅕; and the remainder should be vehicle. Dispersion should continue until the fineness of grind measured on a Hegman gage is between 4 and 8, preferably between 6 and 8. Vehicles used in inks include ethanol, n-propanol, isopropanol, ethyl acetate, propyl acetate, toluene, hexane, dipropylene glycol monomethyl ether, dipropylene glycol monomethyl ether acetate, or mixtures thereof.

In another embodiment of ink preparation, the ink formulation is prepared by combining a vehicle and a soluble polymeric binder, and particles of the composition hereof so that the resulting composition, based on total composition, contains 5-30% of the composition hereof, 4.5-30% of polymer dissolved in the vehicle, and 90.5-50 wt-% vehicle. Optionally, the coating or ink composition may contain, by weight, plasticizer of 0 to 5% and dispersant of 0 to 8%. The ingredients can be combined in any order. Thus, the polymer may be first dissolved in the vehicle followed by additional of particles of the composition hereof which is then dispersed therein; the composition hereof may be in the form of dry particles or a pre-prepared particle dispersion. Alternatively, the particle dispersion may be prepared first followed by addition and dissolution of the polymer.

In another embodiment, an ink formulation is prepared by dissolving a polymeric binder in a mixture of ethyl alcohol and ethyl acetate; the weight of binder is about 90%-150% of the desired amount of the multicopper oxidase enzyme, the oxidizable substrate, and iron. To the solution a portion of the previously prepared dispersion is mixed in containing the desired weight of the multicopper oxidase enzyme, the oxidizable substrate, and iron. To the mixture may be added an amount of plasticizer equal to 1/7 to 1/15 the amount of polymeric binder, preferably ⅛- 1/10 the amount of polymeric binder; and additional vehicle to give the desired ink. The final ink composition, based on total composition, will contain 5%-30% by weight of scavenging particles, preferably 10%-15%; 5%-30% by weight of polymeric binder, preferably 10%-20%; amounts of dispersant and plasticizer based on the scavenger and binder amounts as described above; and vehicle as the remainder.

The following Examples illustrate the invention.

### EXAMPLES

### Materials

Unless otherwise indicated, all materials used in the Examples were obtained from Sigma Chemical Corporation (St. Louis, Mo.).

Myceliophthora thermophilia laccase was obtained from Novozymes (Franklinton, N.C.) as DeniLite® II Base (Item #NS37008) The enzyme was supplied on an inert carrier. The enzyme represented about 2% of the total weight and was washed from the carrier using a buffer (50 mM morpholineethanesulfonic acid, pH 5.5, 1 mM ethylenediamine tetraacetic acid) to yield a solution containing 20 mg/ml enzyme.

Myceliophthora thermophilia laccase was also supplied by Novozymes in a concentrated form (NS44141) containing 95 grams of enzyme per liter of aqeuous solution, and was of sufficient purity to be used directly.

### EXAMPLES 1-3 AND COMPARATIVE EXAMPLES A-D

The purpose of these experiments was to compare the time required to scavenge a fixed volume of oxygen using ink compositions of the invention versus using ink compositions formulated from scavenging compositions of the art.

Calcium ascorbate powder was produced by spray drying a 25% by weight solution of calcium ascorbate in water. Spray drying was done in a 3 ft diameter, 15 ft³ volume, pilot spray dryer (Mobile Minor™, Niro Inc, Columbia, Md.). The dryer was supplied with drying air heated to 228° C. A peristaltic pump (Masterflex, Barnant Co, Barrington, Ill.) was used to meter feed solutions to the spray-drying nozzle. A dual fluid nozzle (SU4, Spraying Systems Co., Chicago, Ill.) supplied with 30 psi N₂ was used to spray slurries into the volume of the dryer. The 75° C. aerosol was discharged to an 8 ft² bag filter where entrained solids were disengaged from the spent drying gas.

A combination of calcium ascorbate and laccase powder was produced by spray drying in the manner described supra, except that 0.25% by weight of the entire solution of laccase enzyme was added to the 25% by weight solution of calcium ascorbate in water employed supra.

The resulting particles were combined with nominal 44 μm diameter iron powder (North American Hoganas) in the amounts shown in Table 1 below, to form the scavenger powder.

Inks were made by adding 10 g of “scavenger powder” which consisted of various combinations of iron, calcium ascorbate, and laccase as listed in Table 1, with 20 g of ethanol. The powder was dispersed by shaking in bottles containing an equal volume of 0.5 millimeter ceramic beads on a gyrotary shaker at 400 rpm for 30 min. To the dispersion so formed was added by pouring 30 g of vehicle containing 25% cellulose acetate (Eastman CAP 504-0.2 and 1% triacetin (Eastman) dissolved in ethyl acetate. The dispersion and vehicle were mixed on a gyrotary shaker at 400 rpm for 30 min.

TABLE 1

| | | | Elapsed time to |
|---|---|---|---|
| | | Substrate or Enzyme | scavenge |
| | Iron Content (g) | Substrate Content (g) | 50 cc/g |
| Example 1 | 0.1 | 9.9 g laccase/ascorbate | 46 hr |
| Example 2 | 0.5 | 9.5 g laccase/ascorbate | 42 hr |
| Example 3 | 1.5 | 8.5 g laccase/ascorbate | 32 hr |
| Comp. Ex A | 0.1 | 9.9 g ascorbate | 115 hr |
| Comp. Ex B | 0.5 | 9.5 g ascorbate | 70 hr |
| Comp. Ex C | 1.5 | 8.5 g ascorbate | 57 hr |
| Comp. Ex D | 0.0 | 10 g laccase/ascorbate | 154 hr |

The resulting inks were drawn down on polyethylene sheet to form dry films approximately 100 microns in thickness which were released from the polyethylene as clean ink films. The ink films were weighed and inserted into test bottles. To provide humidity for activation, about 1 g of DuPont Sontara® SPS™ towel was inserted into each bottle and soaked with approximately 4 g of deionized water, in a manner that ensured no physical contact between the ink film and the wet towel.

### Measurement and Results

Continuous time-course measurements of headspace oxygen concentration were performed with an oxygen analyzer from Sable Systems International. The analyzer consisted of one FC1-FC sensor fuel-cell sensor per vessel, an 8-channel interface to process the output of the sensors, and a computer running ExpeData® software to store the readings. The temperature of the bottles' environment was monitored with a thermocouple and recorded. Each sensor was fitted to a drilled hole in the PTFE cap of a 150-ml pressure vessel (Chemglass model CG-1880-41) and the junction between the sensor and the cap was sealed with Aquaseal® urethane sealant. Oxygen concentration measurements were recorded at 25° C. at five-minute intervals. Oxygen scavenged in cubic centimeters of O₂ per gram of ink film (ccO₂/g) was calculated from the % O₂ measurements using the formula ccO₂/g=(Δ % O₂/100)(bottle volume in ml)/(dry ink film mass in g). Results are shown in Table 1 above.

What is claimed is:

1. A composition comprising:
a non-aqueous liquid vehicle,
a multicopper oxidase enzyme,
an oxidizable substrate,
iron, and
an organic binder polymer dissolved or dispersed in the liquid vehicle, wherein the enzyme, the substrate, and the iron are in particulate form and dispersed in the liquid vehicle.

2. The composition of claim 1 wherein the polymer is dissolved in the vehicle.

3. The composition of claim 1 wherein the polymer is dispersed in the vehicle.

4. The composition of claim 1 wherein the polymer is selected from the group consisting of poly(ethylene-co-vinyl acetate), cellulose acetate, cellulose acetate propionate, cellulose acetate butyrate, poly(ethylene-co-vinyl acetate-co-carbon monoxide), poly(butylmethacrylate), poly(butylene adipate) and mixtures thereof.

5. The composition of claim 1 wherein the vehicle is selected from the group consisting of ethyl acetate, ethanol, toluene, tetrahydrofuran, normal propyl alcohol, normal propyl acetate, normal butyl alcohol, ethylene glycol, and mixtures thereof.

6. The composition of claim 1 wherein the oxidizable substrate is selected from the group consisting of calcium ascorbate, sodium ascorbate, ascorbic acid, ammonium ascorbate, and mixtures thereof.

7. The composition of claim 1 wherein the oxidizable substrate, comprises particles in the range of 1 to 100 micrometers.

8. The composition of claim 1 wherein the oxidizable substrate, comprises particles in the range of 1 to 20 micrometers.

9. The composition of claim 1 wherein the multicopper oxidase is laccase or ascorbate oxidase.

10. The composition of claim 1 wherein one part by weight of the multicopper oxidase enzyme, the oxidizable substrate are combined with 0.05 to 20 parts by weight of the organic polymer.

11. The composition of claim 1 wherein one part by weight of enzyme is combined with 20 to 1000 parts by weight of oxidizable substrate.

12. The composition of claim 1 wherein one part by weight of enzyme is combined with 50 to 500 parts by weight of oxidizable substrate.

13. The composition of claim 1 wherein the multicopper oxidase enzyme, the oxidizable substrate, and iron is present at a concentration in the range of 0.1 to 35 weight % based on total composition.

14. The composition of claim 1 wherein the vehicle is evaporated.

15. The composition of claim 1 further comprising a dispersing agent.
